(19) United States
(12) Patent Application Publication
(10) Pub. No.: US 2020/0022580 A1
(43) Pub. Date: Jan. 23, 2020

(54) IMAGING DEVICE

(71) Applicant: Shimadzu Corporation, Kyoto (JP)

(72) Inventor: Akihiro ISHIKAWA, Kyoto (JP)

(73) Assignee: Shimadzu Corporation, Kyoto (JP)

(21) Appl. No.: 16/039,538

(22) Filed: Jul. 19, 2018

(51) Int. Cl. A61B 5/00; A61M 5/00

(52) U.S. Cl. CPC A61B 5/0071; A61M 5/007; A61B 5/0077

(57) ABSTRACT

An imaging device which can easily recognize the timing of administration of a fluorescent dye through reproduction of recorded images. When indocyanine green is administered, an operator manipulates an input unit so that the input unit sends to a controller a signal indicating that indocyanine green has been administered to a subject. When the controller receives the signal indicating that indocyanine green has been administered to the subject, a light source controller turns a fluorescent light source on, and then off after a lapse of a predetermined time.

### BACKGROUND

The present disclosure relates to an imaging device which irradiates a fluorescent dye administered in a body of a subject with excitation light, and takes an image of fluorescence emitted by the fluorescent dye.

A technique called “near-infrared fluorescence imaging” has been used for angiography in surgery. According to the near-infrared fluorescence imaging, indocyanine green (ICG), which is a fluorescent dye, is administered to an affected area using an injector or any other suitable means. Upon receipt of near-infrared light having a wavelength of about 600 to 850 nm as excitation light, indocyanine green emits near-infrared fluorescence having a wavelength of about 750 to 900 nm. An image of the fluorescence is captured by an image sensor capable of detecting the near-infrared light, and is shown on a display unit such as a liquid crystal display panel. According to the near-infrared fluorescence imaging, blood vessels and lymphatics at the depth of about 20 mm from the body surface can be observed.

Further, attention has recently been paid to a technique of fluorescence-labeling a tumor for the purpose of surgery navigation. As a fluorescent marker used for the fluorescence-labeling of the tumor, 5-ALA-aminolevulinic acid is used. When administered to a subject, 5-ALA-aminolevulinic acid (will be hereinafter abbreviated as “5-ALA”) is metabolized by protoporphyrin IX (PpIX), which is one of the fluorescent dyes. PpIX specifically accumulates in cancer cells. When visible light having a wavelength of about 410 nm is applied to PpIX, which is a metabolite of 5-ALA, PpIX emits red visible light having a wavelength of about 630 nm as fluorescence. Thus, the cancer cells can be identified through the observation of the fluorescence from PpIX.

International Patent Publication No. 2009/139466 discloses a data collection method. In this method, an intensity distribution image of near-infrared fluorescence obtained through excitation light irradiation of a subject organ of a living body administered with indocyanine green is compared with a cancer lesion distribution image obtained through X-ray irradiation, nuclear magnetic resonance, or ultrasonography performed on the subject organ before the administration of indocyanine green. Then, data of a region which is detected in the intensity distribution image of the near-infrared fluorescence, but not in the cancer lesion distribution image is collected as data of a sub-lesion region of cancer.

### SUMMARY

In the imaging device configured to take an image of the fluorescence from the fluorescent dye injected in the body, the fluorescence from the subject and images of the subject under visible light are simultaneously recorded as a video, which is reproduced by a video recorder. Thus, according to a conventional imaging device, images taken at a predetermined frame rate are recorded and reproduced as a video, so that the courses of the blood vessels and the lymphatics after the administration of the fluorescent dye such as ICG can be observed, and a region of a cancer lesion can be identified, in a bright external lighting environment.

Such recorded data can be used not only for reference purposes, but also for obtaining new findings through analyses. For example, in a time intensity curve (TIC) analysis based on a curve of time-varying changes in signal of a region of interest (ROI), time taken until the pixel value of the ROI reaches the peak is obtained so that imaging time of the fluorescent dye such as indocyanine green can be quantitatively evaluated.

So far, during the video reproduction and processing, it has been checked only visually at what timing the administration of the fluorescent dye such as indocyanine green started. Therefore, if the administration of the fluorescent dye is performed outside the imaging field, the timing of the administration cannot be recognized through the reproduced video only.

In view of the foregoing, the present disclosure has been achieved to provide an imaging device which can easily recognize the timing of the administration of the fluorescent dye through the reproduction of recorded images.

A first aspect of the present disclosure is directed to an imaging device which includes: an excitation light source which irradiates a subject with excitation light for exciting a fluorescent dye administered to the subject; a shooting unit which shoots fluorescence emitted from the fluorescent dye irradiated with the excitation light to obtain a fluorescence image; and an image storage which stores the fluorescence image as a video. The imaging device includes: a fluorescent light source which irradiates the subject with light having a wavelength corresponding to the fluorescence; and a light source controller which turns the fluorescent light source on only for a predetermined time upon receipt of a signal indicating that the fluorescent dye has been administered to the subject.

A second aspect of the present disclosure is directed to an imaging device which includes: an excitation light source which irradiates a subject with excitation light for exciting a fluorescent dye administered to the subject; a visible light source which irradiates the subject with visible light; a shooting unit which shoots fluorescence emitted from the fluorescent dye irradiated with the excitation light, and visible light reflected from a surface of the subject to obtain a fluorescence image and a visible light image; and an image storage which stores the fluorescence image and the visible light image as a video. The imaging device includes a light source controller which changes an intensity of the visible light from the visible light source only for a predetermined time upon receipt of a signal indicating that the fluorescent dye has been administered to the subject.

A third aspect of the present disclosure is an embodiment of the first or second aspect. In the third aspect, the imaging device further includes: an input unit which is manipulated by an operator to send the signal indicating that the fluorescent dye has been administered to the subject.

A fourth aspect of the present disclosure is an embodiment of the first or second aspect. In the fourth aspect, the signal indicating that the fluorescent dye has been administered to the subject is received from an injector which injects the fluorescent dye into the subject.

According to the first aspect, the fluorescent light source is lit only for a predetermined time after the administration of the fluorescent dye to the subject. Thus, an operator can recognize that the fluorescent dye has been administered by observing the fluorescence image. Consequently, the timing of the fluorescent dye administration can easily be recognized through the reproduction of the recorded video.

According to the second aspect, the intensity of the visible light emitted from the visible light source is changed only for a predetermined time after the administration of the fluorescent dye to the subject. Thus, an operator can recognize that the fluorescent dye has been administered by observing the fluorescence image. Consequently, the timing of the fluorescent dye administration can easily be recognized through the reproduction of the recorded video.

According to the third aspect, the input unit is manipulated by an operator so that the signal indicating that the fluorescent dye has been administered to the subject can be sent from the input unit.

According to the fourth aspect, the injector which injects the fluorescent dye to the subject can send the signal indicating that the fluorescent dye has been administered to the subject.

### BRIEF DESCRIPTION OF THE DRAWINGS

FIG. 1 is a perspective view illustrating an imaging device of the present disclosure.

FIG. 2 is a side view illustrating the imaging device of the present disclosure.

FIG. 3 is a plan view illustrating the imaging device of the present disclosure.

FIG. 4 is a perspective view of a lighting/shooting unit 12.

FIG. 5 is a schematic front view of the lighting/shooting unit 12.

FIG. 6 is a schematic view of a camera 21 of the lighting/shooting unit 12.

FIG. 7 is a block diagram illustrating a major control system of the imaging device of the present disclosure.

FIG. 8 is a schematic view illustrating how videos are shown on a monitor 15.

FIG. 9 is a graph illustrating time intensity curves shown on the monitor 15.

FIG. 10 is a graph illustrating time intensity curves shown on the monitor 15.

### DETAILED DESCRIPTION

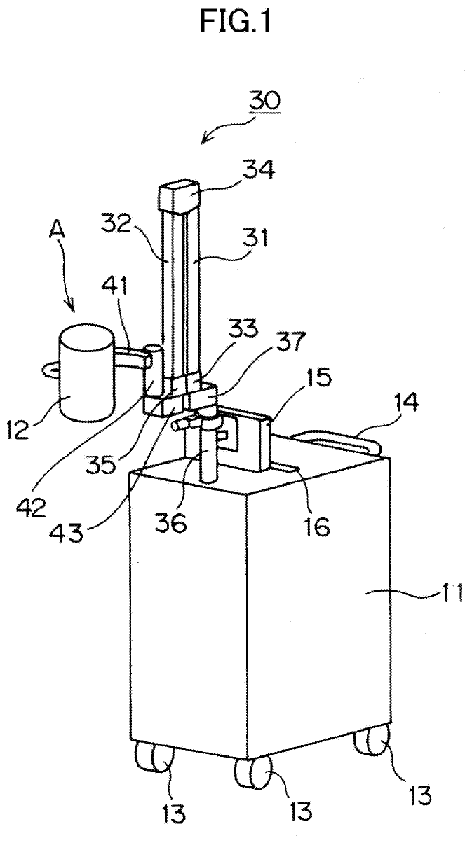

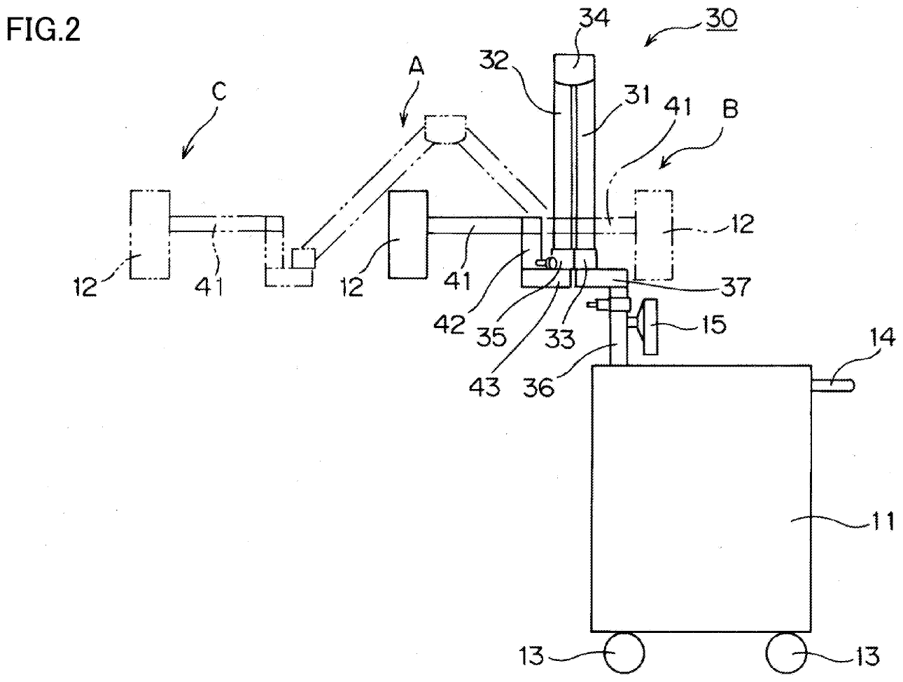

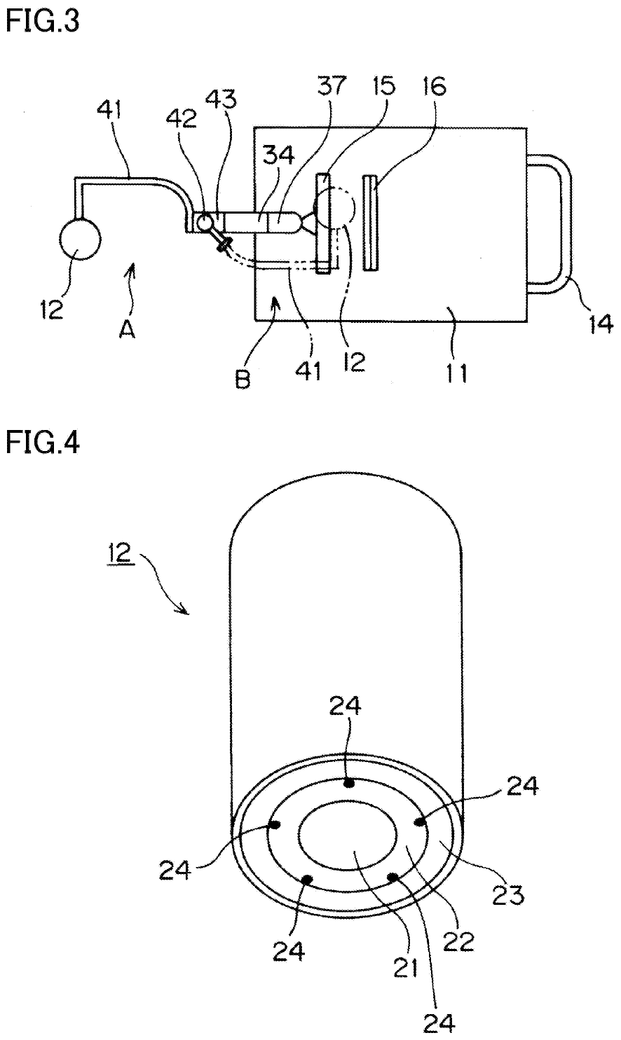

Embodiments of the present disclosure will be described in detail with reference to the drawings. FIG. 1 is a perspective view illustrating an imaging device of the present disclosure. FIG. 2 is a side view illustrating the imaging device of the present disclosure. FIG. 3 is a plan view illustrating the imaging device of the present disclosure. The disclosed imaging device irradiates indocyanine green, which is a fluorescent dye injected into a body of a subject, with excitation light, and shoots fluorescence emitted from indocyanine green. The imaging device includes a wagon 11 with four wheels 13, an arm mechanism 30 disposed on a portion of a top surface of the wagon 11 toward the front in a traveling direction of the wagon 11 (toward the left in FIGS. 2 and 3), a lighting/shooting unit 12 provided for the arm mechanism 30 via a sub-arm 41, and a monitor 15. The “front in the traveling direction” of the wagon 11 will be hereinafter simply referred to as the “front” of the wagon 11. A handle 14 used to move the wagon 11 is attached to a rear side of the wagon 11 in the traveling direction. A recess 16 is formed at the top surface of the wagon 11 so that a remote control for operating the imaging device from a distance can fit therein.

The arm mechanism 30 is disposed on the front portion of the wagon 11. The arm mechanism 30 includes a first arm member 31 which is coupled via a hinge 33 to a support 37 arranged on a column 36 standing upright on the front portion of the wagon 11. The first arm member 31 is able to swing with respect to the wagon 11 via the column 36 and the support 37 by the action of the hinge 33. The monitor 15 is attached to the column 36.

A second arm member 32 is coupled to an upper end of the first arm member 31 via a hinge 34. The second arm member 32 is able to swing with respect to the first arm member 31 by the action of the hinge 34. In this configuration, the first and second arm members 31 and 32 are able to take a shooting position as indicated by reference character C and phantom lines in FIG. 2, and a standby position as indicated by reference character A and solid lines in FIGS. 1 to 3. In the shooting position, the first and second arms 31 and 32 form a predetermined angle around the hinge 34 coupling the first and second arm members 31 and 32. In the standby position, the first and second arm members 31 and 32 are adjacent to each other.

A support 43 is coupled to a lower end of the second arm member 32 via a hinge 35. The support 43 is able to swing with respect to the second arm member 32 by the action of the hinge 35. The support 43 supports a rotation shaft 42. The sub-arm 41 supporting the lighting/shooting unit 12 rotates about the rotation shaft 42 disposed at a tip end of the second arm member 32. Thus, through the rotation of the sub-arm 41, the lighting/shooting unit 12 moves between a front position and a rear position with respect to the arm mechanism 30 in the traveling direction of the wagon 11. The front position, which corresponds to the shooting position or the standby position, is indicated by reference character A and solid lines in FIGS. 1 to 3, or reference character C and phantom lines in FIG. 2. The rear position, which is a position during the movement of the wagon 11, is indicated by reference character B and phantom lines in FIGS. 2 and 3.

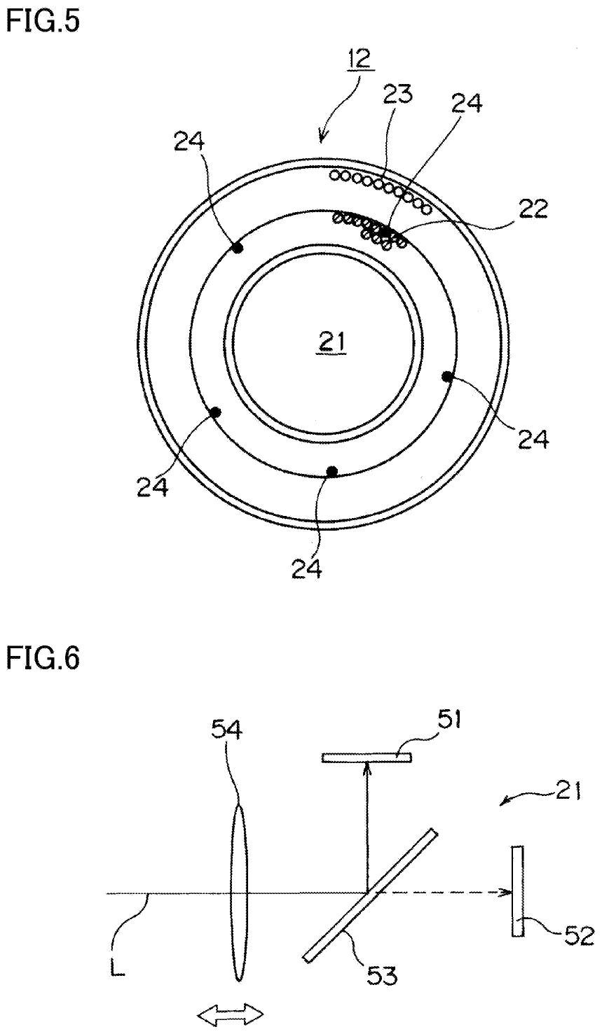

FIG. 4 is a perspective view of the lighting/shooting unit 12. FIG. 5 is a schematic front view of the lighting/shooting unit 12.

The lighting/shooting unit 12 includes a camera 21 which can detect near-infrared light and visible light, a visible light source 22 comprised of a plurality of LEDs disposed on the outer periphery of the camera 21, an excitation light source 23 comprised of a plurality of LEDs disposed on the outer periphery of the visible light source 22, and a fluorescent light source 24 comprised of a plurality of LEDs disposed in the plurality of LEDs of the visible light source 22. The visible light source 22 emits visible light. The excitation light source 23 emits near-infrared light having a wavelength of 760 nm as excitation light for exciting indocyanine green. The fluorescent light source 24 emits near-infrared light having a wavelength of 810 nm corresponding to the wavelength of fluorescence emitted from indocyanine green. In FIG. 5, some of the LEDs of the visible light source 22 and the LEDs of the excitation light source 23 are omitted. The wavelength of light emitted from the excitation light source 23 is not limited to 760 nm as long as the light can excite indocyanine green. The wavelength of light emitted from the fluorescent light source 24 is not limited to 810 nm. The light may have any wavelength as long as the wavelength is close to the wavelength of fluorescence emitted from indocyanine green and the light can be captured by a fluorescence image sensor 52 which will be described later. In this specification, the wavelength corresponding to the wavelength of the fluorescence means a wavelength which is approximate to the wavelength of the fluorescence, and can be captured by the fluorescence image sensor 52.

In this embodiment, the camera 21, the visible light source 22, the excitation light source 23, and the fluorescent light source 24 are integrated into the lighting/shooting unit 12. Alternatively, some or all of the camera 21, the visible light source 22, the excitation light source 23, and the fluorescent light source 24 may be separately arranged.

FIG. 6 is a schematic view of the camera 21 of the lighting/shooting unit 12.

The camera 21 includes a moving lens 54 which reciprocates for focusing, a wavelength selection filter 53, a visible light image sensor 51, and a fluorescence image sensor 52. The visible light image sensor 51 and the fluorescence image sensor 52 are comprised of CMOS or CCDs. Visible light and fluorescence coaxially entering the camera 21 along its optical axis L pass through the moving lens 54 as a component of a focusing mechanism, and reach the wavelength selection filter 53. The visible light that has entered coaxially together with the fluorescence is reflected by the wavelength selection filter 53, and enters the visible light image sensor 51. The fluorescence that has entered coaxially together with the visible light passes through the wavelength selection filter 53, and enters the fluorescence image sensor 52. At this time, by the action of the focusing mechanism including the moving lens 54, the visible light is focused on the visible light image sensor 51, while the fluorescence is focused on the fluorescence image sensor 52.

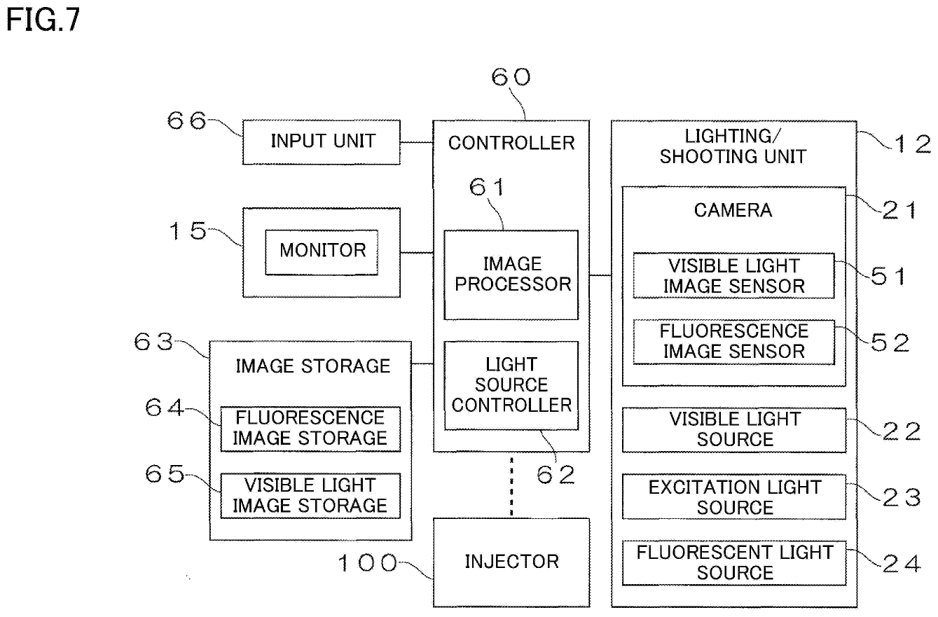

FIG. 7 is a block diagram illustrating a major control system of the imaging device of the present disclosure.

The imaging device includes a controller 60 comprised of a CPU which executes logical operations, a ROM which stores programs necessary for controlling the device, and a RAM which temporarily stores data during control. The controller 60 entirely controls the imaging device. The controller 60 includes an image processor 61 which executes various types of image processing on fluorescence images and visible light images. Further, the controller 60 includes a light source controller 62 which can perform on/off control of the visible light source 22, the excitation light source 23, and the fluorescent light source 24, and control the intensity of irradiation from these light sources.

The controller 60 is connected to an input unit 66 through which an operator can enter various information items. The various information items may include information that indocyanine green has been administered to a subject who will be described later. The controller 60 is connected to the monitor 15. The input unit 66 may be provided for a remote control used to operate the imaging device from a distance. If the monitor 15 is a touch panel, the input unit 66 may be shown on a screen of the monitor 15, or disposed on the wagon 11.

Further, the controller 60 is connected to the lighting/shooting unit 12 including the camera 21, the visible light source 22, the excitation light source 23, and the fluorescent light source 24. The controller 60 is also connected to an image storage 63 which stores images taken by the camera 21. The image storage 63 includes a fluorescence image storage 64 which stores fluorescence images, and a visible light image storage 65 which stores visible light images. The fluorescence image storage 64 and the visible light image storage 65 may be replaced with a synthetic image storage which stores images obtained by synthesis (fusion) of the visible light images and the fluorescence images.

The controller 60 is also connected to an injector 100 for injecting indocyanine green into the subject.

It will be described below an imaging operation according to the first embodiment performed in surgery of a subject using the imaging device of the above-descried configuration.

In the surgery of a subject, an operator holds the handle 14 to move the wagon 11 to bring the imaging device to a site of surgery. Once the imaging device is properly set, an affected part of the subject is irradiated with visible light emitted from the visible light source 22, and near-infrared light having a wavelength of 760 nm emitted from the excitation light source 23 as the excitation light for exciting indocyanine green. Then, the affected part and its vicinity are shot by the camera 21 of the lighting/shooting unit 12. The visible light and the excitation light may be emitted to the affected part simultaneously. Alternatively, the visible light and the excitation light may be emitted in a pulsed manner at different intervals, and the visible light image sensor 51 and the fluorescence image sensor 52 shown in FIG. 6 may capture images concurrently with the emission of the visible light and the excitation light.

Then, the injector 100 shown in FIG. 7 administers an injection of indocyanine green to the subject. In one embodiment, when indocyanine green is administered, the operator manipulates the input unit 66 to send a signal indicating that indocyanine green has been administered to the subject from the input unit 66 to the controller 60. In another embodiment, the signal indicating that indocyanine green has been administered to the subject is sent from the injector 100 to the controller 60.

Once the controller 60 receives the signal indicating that indocyanine green has been administered to the subject, the light source controller 62 shown in FIG. 7 turns the fluorescent light source 24 on, and then off after a lapse of a predetermined time.

Images of the visible light reflected from the subject are captured as visible light images by the visible light image sensor 51 of the camera 21. Being irradiated with the near-infrared light having a wavelength of 760 nm emitted from the excitation light source 23, indocyanine green administered to the subject's body emits fluorescence in an infrared region having a peak around 810 nm. Images of the fluorescence are captured as fluorescence images by the fluorescence image sensor 52 of the camera 21. The visible light images and the fluorescence images are displayed on the monitor 15 as videos, and respectively stored in the fluorescence image storage 64 and visible light image storage 65 of the image storage 63 as videos. The visible light images and the fluorescence images are stored as video files in a lossy compression format for display, and also as video files in a lossy compression or non-compression format for TIC analysis.

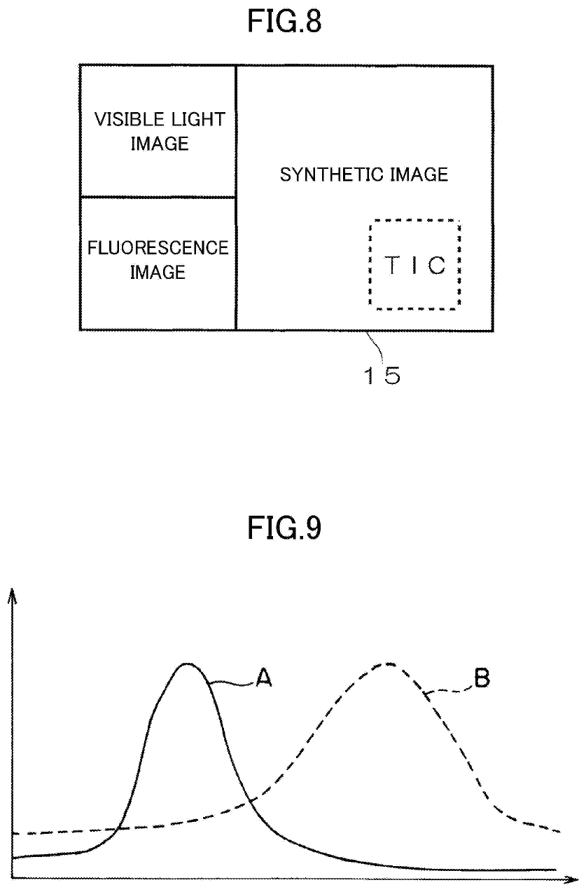

FIG. 8 is a schematic view illustrating how videos are shown on the monitor 15.

The monitor 15 shows the visible light image, the fluorescence image, and a synthetic image of the visible light image and the fluorescence image. A TIC, which is a curve of time-varying changes in signal of the ROI (changes in pixel value), is shown over a portion of the synthetic image. The visible light image, the fluorescence image, the synthetic image, and the TIC are shown not only on the monitor 15, but also on a large display unit provided separately from the imaging device.

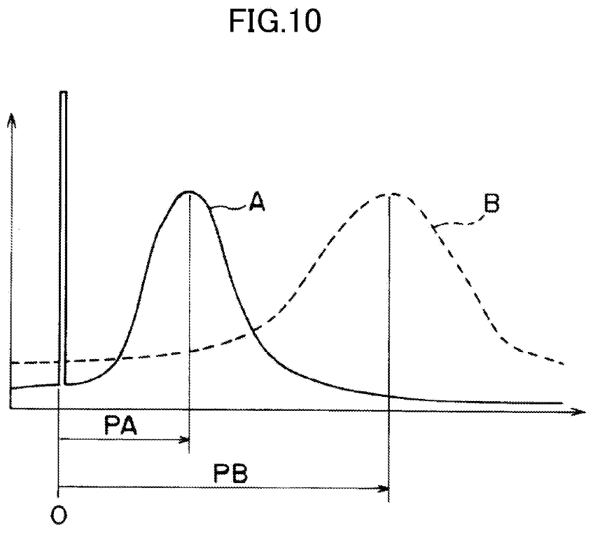

FIGS. 9 and 10 are graphs illustrating the TIC shown on the monitor 15. In these graphs, a vertical axis represents a pixel value of the fluorescence image, and a horizontal axis time. Reference characters A and B indicate curves of the changes in pixel value in different sites of the ROI.

In the TIC analysis, time taken until the pixel value of the ROI reaches the peak is obtained so that imaging time of indocyanine green can be quantitatively evaluated. If the fluorescence images stored in the fluorescence image storage 64 are analyzed, a TIC indicating the time-varying changes in signal of the ROI is displayed as shown in FIG. 9. In this case, it has been impossible to recognize at what timing the administration of indocyanine green started.

In contrast, the imaging device of the present disclosure is configured such that once the controller 60 receives the signal indicating that indocyanine green has been administered to the subject, the light source controller 62 turns the fluorescent light source 24 on, and then off after a lapse of a predetermined time. Thus, as shown in FIG. 10, while the fluorescent light source 24 is on, the timing of the administration of indocyanine green can easily be recognized because the pixel value of the fluorescence image temporarily shows a steep rise. Therefore, in each site, time PA or PB taken until the fluorescence emitted from indocyanine green reaches the peak after the administration of indocyanine green can easily be recognized.

A second embodiment of the present disclosure will be described below.

According to the imaging device of the first embodiment described above, the light source controller 62 turns the fluorescent light source 24 on only for a predetermined time after the receipt of the signal indicating that indocyanine green has been administered to the subject. In contrast, according to an imaging device of the second embodiment, the intensity of the visible light from the visible light source 22 is changed only for a predetermined time after the receipt of the signal indicating that indocyanine green has been administered to the subject.

Specifically, in the second embodiment, once the controller 60 receives the signal indicating that indocyanine green has been administered to the subject, the light source controller 62 increases the intensity of the visible light from the visible light source 22, and then resets the intensity to the original value after a lapse of a predetermined time. As a result, the visible light image in a visible light image region of the monitor 15 of FIG. 8 is temporarily brightened. Perceiving the brightened image, the operator can recognize the administration timing of indocyanine green.

Also in the second embodiment, the administration timing of indocyanine green may be recognized based on the TIC obtained from the visible light image as shown in FIG. 10. Upon receipt of the signal indicating that indocyanine green has been administered to the subject, the light source controller 62 may lower the intensity of the visible light from the visible light source 22, instead of increasing it.

It has been described in the foregoing embodiment that indocyanine green is used as the fluorescent dye, and irradiated with near-infrared light of about 600 to 850 nm as the excitation light so that fluorescence in a near-infrared region having a peak around 810 nm is emitted from indocyanine green. Alternatively, light other than the near-infrared light may be used.

Further, indocyanine green used as the fluorescent dye may be replaced with other fluorescent dye such as 5-ALA mentioned above.

What is claimed is:

1. An imaging device comprising:
an excitation light source which irradiates a subject with excitation light for exciting a fluorescent dye administered to the subject;
a shooting unit which shoots fluorescence emitted from the fluorescent dye irradiated with the excitation light to obtain a fluorescence image; and
an image storage which stores the fluorescence image as a video,
the imaging device further comprising a fluorescent light source which irradiates the subject with light having a wavelength corresponding to the fluorescence;
a light source controller which turns the fluorescent light source on only for a predetermined time upon receipt of a signal indicating that the fluorescent dye has been administered to the subject.

2. An imaging device comprising:
an excitation light source which irradiates a subject with excitation light for exciting a fluorescent dye administered to the subject;
a visible light source which irradiates the subject with visible light;
a shooting unit which shoots fluorescence emitted from the fluorescent dye irradiated with the excitation light, and visible light reflected from a surface of the subject to obtain a fluorescence image and a visible light image;
an image storage which stores the fluorescence image and the visible light image as a video,
the imaging device further comprising a light source controller which changes an intensity of the visible light from the visible light source only for a predetermined time upon receipt of a signal indicating that the fluorescent dye has been administered to the subject.

3. The imaging device of claim 1, further comprising:
an input unit which is manipulated by an operator to send the signal indicating that the fluorescent dye has been administered to the subject.

4. The imaging device of claim 1, wherein the signal indicating that the fluorescent dye has been administered to the subject is received from an injector which injects the fluorescent dye into the subject.

5. The imaging device of claim 2, further comprising:
an input unit which is manipulated by an operator to send the signal indicating that the fluorescent dye has been administered to the subject.

6. The imaging device of claim 2, wherein the signal indicating that the fluorescent dye has been administered to the subject is received from an injector which injects the fluorescent dye into the subject.
